(19) United States
(12) Patent Application Publication
(10) Pub. No.: US 2010/0191113 A1
(43) Pub. Date: Jul. 29, 2010

(54) SYSTEMS AND METHODS FOR ULTRASOUND IMAGING WITH REDUCED THERMAL DOSE

(75) Inventors: Christopher Robert Hazard, Niskayuna, NY (US); Feng Lin, Niskayuna, NY (US); Mirsaid Seyed-Bolorforosh, Albany, NY (US); Kenneth Wayne Rigby, Clifton Park, NY (US)

Correspondence Address: GENERAL ELECTRIC COMPANY;GLOBAL RESEARCH, ONE RESEARCH CIRCLE, BLDG. K1-3A59, NISKAYUNA, NY, 12309, US

(73) Assignee: GENERAL ELECTRIC COMPANY, Schenectady, NY (US)

(21) Appl. No.: 12/360,881

(22) Filed: Jan. 28, 2009

(51) Int. Cl. A61B 8/14; G01N 29/04

(52) U.S. Cl. 600/443; 73/596; 600/459

(57) ABSTRACT

An ultrasound imaging method is provided. The method includes identifying a plurality of locations within a region of interest, delivering a pulse sequence to two or more of the plurality of locations in a determined order, wherein the pulse sequence comprises a pushing pulse, and a tracking pulse, and applying a motion correction sequence to each of the plurality of locations where the pulse sequence is delivered.

### BACKGROUND

Embodiments of the invention relate to ultrasound imaging, and more particularly to acoustic radiation force impulse (ARFI) imaging.

Tissue stiffness has been shown to be a marker of disease. For example, some cancerous tissues are stiffer than the normal surrounding tissues. Treatments for certain conditions, such as ablation, also create stiffer regions of tissue. Acoustic radiation force impulse imaging refers to a method of pushing tissue with a relatively long and high intensity acoustic pulse and then tracking the displacement of the tissue. The ARFI imaging method gives information about the stiffness of the tissue.

The long and high intensity pulses used in ARFI can create thermal issues in the imaging system as well as in the object that is being imaged. Typically, the heating created during ARFI can be divided into transducer heating and tissue heating.

A portion of the electrical energy used to excite the crystals/ceramics or other materials in the transducer is lost in the form of heat, thereby causing transducer heating. In ARFI, transducer heating is a concern because of the high amplitude and long duration pulses required. The International Electrotechnical Commision (IEC) requires that the temperature of an ultrasound probe surface which contacts the patient never exceed 43° C. (IEC 60601-1). Generally, the transducer heating can be reduced by modifying the lens material or design, by including thermal management features in the acoustic stack, and through the use of active cooling devices.

In contrast, tissue heating related to ARFI pushing pulses is a more difficult problem to address. Although the rise in temperature inside a body being imaged can be monitored through ultrasound based and MRI based methods for remote temperature sensing, these are cumbersome, unreliable, or expensive. Typically, temperature rise in the body needs to be estimated based on models and assumptions. Even if the temperature can be monitored, there is little that can be done to remove the heat. Therefore, ways of reducing the amount of energy deposited in the tissue are required.

### BRIEF DESCRIPTION

In one embodiment, an ultrasound imaging method is provided. The method includes identifying a plurality of locations within a region of interest, delivering a pulse sequence to two or more of the plurality of locations in a determined order, wherein the pulse sequence comprises a pushing pulse, and a tracking pulse, and applying a motion correction sequence to each of the plurality of locations where the pulse sequence is delivered.

In another embodiment, an ultrasound imaging system is provided. The system includes a transducer array configured to deliver an ARFI pulse sequence to a plurality of locations in a region of interest, wherein the ARFI pulse sequence comprises a tracking pulse, and a pushing pulse, a controller for controlling delivery of the ARFI pulse sequences to the plurality of locations in a determined order, or for controlling delivery of a motion correction sequence, and applying the motion correction sequence to each of the plurality of locations where the pulse sequence is delivered, and a signal processing unit for processing received data from the plurality of locations in response to the plurality of ARFI pulse sequences, and the motion correction sequence.

### DRAWINGS

These and other features, aspects, and advantages of the present invention will become better understood when the following detailed description is read with reference to the accompanying drawings in which like characters represent like parts throughout the drawings, wherein:

FIGS. 1-2 are schematic representations of an imaging method for reducing heat or thermal dose in a region of interest, in accordance with embodiments of the present technique;

FIGS. 3-5 are schematic representations of an example application of a 2D cross correlation algorithm to achieve motion correction, in accordance with embodiments of the present technique;

FIG. 6 is a flow chart illustrating an example algorithm employed to reduce tissue heating in the region of interest, in accordance with embodiments of the present technique;

FIGS. 7-9 are schematic representations of examples of delivering the pulse sequences to the plurality of locations, in accordance with embodiments of the present technique; and

FIG. 10 is a schematic representation of an ultrasound imaging system for reducing heat or thermal dose in a region of interest, in accordance with embodiments of the present technique.

### DETAILED DESCRIPTION

In certain embodiments, an ultrasound imaging method and system are provided. The imaging method of the present technique may facilitate reduction of heat in a region of interest (ROI) that is being imaged. The method includes identifying a plurality of locations within a region of interest, delivering a pulse sequence to two or more of the plurality of locations in a determined order. The plurality of locations may be selected manually or by employing an automated algorithm. The ROI is composed of a series of vectors or beams. As used herein, vectors refer to locations of pushing and tracking that are used to make an ARFI image. Typically, in ultrasound imaging, the ROI is repeatedly interrogated by firing the same group of vectors multiple times and displaying the results as a series of images that change over time. Usually, the locations of the vectors are held constant from frame to frame. As used herein, a frame refers to the collection of vectors that makes up an ROI that are fired at a similar time. In one embodiment, the plurality of locations may exist in a single frame. The order in which the vectors are fired is chosen to minimize the heat delivered, however, the same locations are used from frame to frame. In another embodiment, the plurality of locations may be inter-grid locations to facilitate reduction of thermal dose. In this embodiment, not only is the order of the vectors different, but the locations of the vectors may also vary from frame to frame. For example, the second frame of firing may fire vectors at locations that would be between the locations of the firings in a first frame. This allows, the location of peak energy deposition to be changed from frame to frame.

The determined order for which the pulse sequences are to be delivered may be selected based on a cost function, for example, which may be designed to minimize the total amount of heat or thermal dose for a given location. The pulse sequence may be delivered to a particular location one or more times. In embodiments where the pulse sequence is delivered to the locations only once, the pulse sequence may include a reference pulse, a pushing pulse and a tracking pulse. Whereas, in embodiments where the pulse sequences are delivered to the plurality of locations two or more times, the different pulse sequences may or may not contain the reference pulse. In one embodiment where the pulse sequences do not include the reference pulse, the reference pulse may be delivered to the location initially when the pulse sequence is being delivered for the first time to the location, and the subsequent pulse sequences may be delivered without any reference pulse. In another embodiment where the pulse sequences comprise the reference pulse, the reference pulse may be delivered with each pulse sequence.

In certain embodiments, a motion correction sequence may be applied to each of the plurality of locations where the pulse sequence is delivered. The motion correction sequence takes into account any involuntary motion of the imaging object, such as a patient, or the transducer probe, or the person (such as the sonographer or the physician) performing the imaging. The transducer array may be a one-dimensional, or a two-dimensional array. The motion correction sequence may be delivered between the pulse sequences. The motion correction sequence may be delivered immediately before or after delivering a pulse sequence to a particular location. In one embodiment, the motion correction sequence may include a B-mode sequence. The B-mode sequence may be a complete B-mode sequence or a partial B-mode sequence, or a combination of a complete and a partial B-mode sequence.

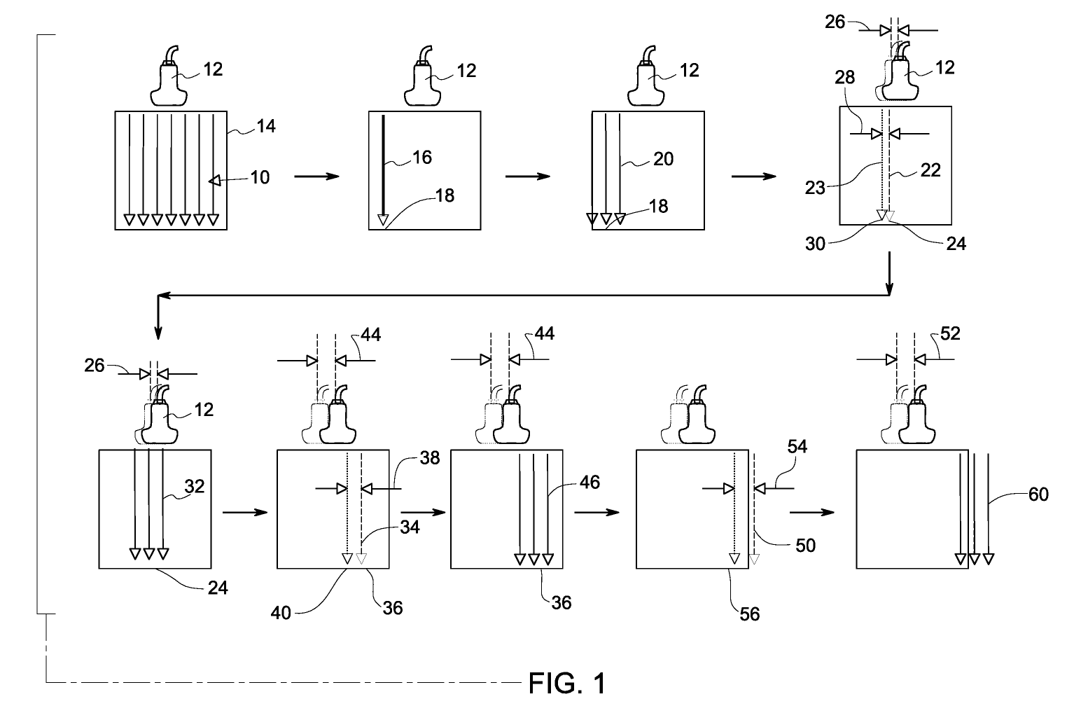

FIG. 1 illustrates an example of an imaging method that may be employed to reduce heat or thermal dose in a region of interest. Initially, a plurality of locations is identified within the region of interest. As illustrated in FIG. 1, an original frame B-mode pulse sequence 10 is delivered using a transducer probe 12 to a region of interest in a single frame 14 having a plurality of locations. The original frame B-mode image thus acquired works as a reference image for motion correction of the subsequent images. The original frame B-mode image may provide a baseline for motion correction during subsequent imaging. Next, a first pulse sequence represented by the vector 16 may be delivered to a first location 18.

Subsequently, a partial B-mode sequence represented by the three vectors 20 may be delivered in and around the same location 18 where the first pulse sequence 16 is delivered. The image obtained from the partial B-mode sequence, such as the sequence 20, may be correlated with the image obtained from the original frame B-mode pulse sequence 10 to determine the location of the pulse sequence, such as the pulse sequence 16 in real space. Also, all the subsequent firings of the pulse sequences may be aligned with the original frame B-mode image. By determining the location of the pulse sequence in the real space, the subsequent firing locations of the pulse sequences may be corrected for the newly characterized motion. In addition, an algorithm may be applied to create an image (for example, a two dimensional image) by interpolating to a grid (for example, a two dimensional grid) or sector given the known real space location of the pulse sequences. Generally, the pulse sequence (pushing pulse and a tracking pulse) is a long series of firings, and the additional partial B-mode sequence fired immediately before or after the pulse sequence utilizes only a fraction of time used by the pulse sequence.

In one embodiment, the size of the partial B-mode is selected based on the determined level of a thermal dose, the imaging time, or the motion of tissues in at least one of the plurality of locations. As used herein, the term “size of the partial B-mode” refers to the lateral width of the partial B-mode image and the term “density of the partial B-mode” refers to the number of vectors in the partial B-mode. The size and density of the partial B-mode sequence may be selected based on a number of factors. For example, a large partial B-mode sequence provides more data for correlation than a small partial B-mode sequence resulting in better motion correction. However, as the partial B-mode sequence gets larger there is less space to slide the window of the region of interest in the original B-mode sequence, hence, the range of motion is reduced. Also, as the size of the partial B-mode sequence gets larger the amount of time taken to collect the data increases. In addition, heating from the partial B-mode transmissions becomes larger. Additionally, if the motion of the ROI is not rigid, as the size of the partial B-mode sequence gets larger and a comparison is made against a now warped version of the original B-mode, a poor correlation is obtained. If the motion is a simple translation of the entire field of view, then correlation processing will track the motion well. However, if the motion is more complex and different parts of the tissue move by different amounts or in different directions, then the correlation processing is less effective. A smaller partial B-mode needs the motion to be constant over a smaller region and thus is less affected by the overall non-rigid motion.

Next, a second pulse sequence represented by the vector 22 may be delivered at a second location 24 that is shifted by a distance 28 from the desired location 30. This shift in the desired location 30 and the actual location 24 may be caused due to the inadvertent shift 26 in the position of the transducer probe 12, for example.

A partial B-mode sequence represented by the three vectors 32 is then delivered in and around the actual position 24. Next, a third pulse sequence represented by the vector 34 may be then delivered to an actual location 36, which may be shifted by a distance 38 from the desired location 40 due to an involuntary shift 44 in the position of the transducer probe 12, for example. A partial B-mode sequence represented by the three vectors 46 may be delivered in and around the actual location 36.

Subsequently, a fourth pulse sequence represented by the vector 50 may be delivered to a location that may fall outside the frame 14 due to a further shift 52 in the probe position. The shift 52 in the probe position results in a shift 54 of the desired location 56 for delivering the pulse sequence 50. Due to this shift 52 of the probe 12, the partial B-mode sequence 60 may be delivered in and around the actual location. Accordingly, at least a portion of the B-mode sequence 60 may fall outside the frame 14.

In some embodiments, each of the first, second, third and fourth pulse sequences 16, 22, 34 and 50, respectively, include a pushing pulse and a tracking pulse. In other embodiments, the first pulse sequence includes a reference pulse in addition to the pushing pulse and the tracking pulse, whereas, the remaining pulse sequences include only the pushing pulse and the tracking pulse. In some embodiments, all the pulse sequences may include a reference pulse, a pushing pulse and a tracking pulse. The reference pulse may be delivered to detect an initial position of the location, the pushing pulse may be delivered to a location to displace the tissues of the location to a first displaced position, and the tracking pulse may be delivered to a location to detect the first displaced position of the target region. The pushing pulse may be a single pulse or a combination of pulses. Similarly, the tracking pulse may either be a single pulse or a series of pulses.

The delivery of the pulse sequences to a particular location may be separated in time. This separation may be determined by the time required to allow the tissue to settle back into a particular state, which may either be the initial state, or a slightly displaced state. In one embodiment, additional time, also referred to as cooling time, may be added between delivering the pulse sequences to facilitate reduction of tissue heating. Typically, the amplitude and length of the pushing pulse determines the pace at which the tissue heats up. In embodiments, where a single frame of an image is desired, it may be possible to acquire the image without significant heating of the tissue. However, if multiple frames are desired, for example, to track change over time, or to provide averaging ability, then cumulative heating may result. In one embodiment, the cooling time between the individual pulse sequences may be adjusted depending on the number of frames required for the application. For example, if a single or small number of frames is required, the cooling time may be smaller, thereby allowing for faster collection. Whereas, if a large number of frames is required, the cooling time between the pulse sequences may be increased to reduce the cumulative heating effect.

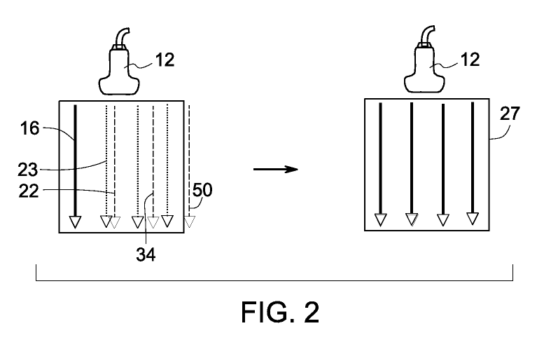

FIG. 2 represents the acquired image formed by vectors 16, 22, 34 and 50, and the acquired image formed by vectors 16, 23, 35 and 51, and the scan sequence with motion correction. As illustrated by reference number 27, the desired and actual vectors of the pulse sequences overlap after application of a motion correction sequence. Initially when the probe position is not changed, the actual and desired locations for firing the pulse sequences 16 is the same. If scan conversion is done to obtain the image using the pulse sequences without using the motion correction sequence, such as sequence 10, 20, 32, 46 and 60, the obtained image may be distorted. However, by providing a motion correction sequence, the correction may be made for the involuntary motion of the probe, or the object being imaged, or the technician performing the imaging. The partial B-mode images captured by sequences 20, 32, 46 and 60 may be registered to the original frame B-mode image of the original frame B-mode pulse sequence 10. The realignment of the images based on the motion correction sequence may be performed by employing algorithms. Non-limiting examples of such algorithms may include 2D block matching, 3D block matching, 1D cross correlation, 2D cross correlation, 3D cross correlation, sum-of-absolute differences, sum of square difference, and minimum entropy.

The illustrated embodiment of FIGS. 1-2 is described with regard to a single frame. However, a similar method may be applied for more than one frame. In the case of more than one frame, the same method may be repeated for each frame in a series of frames. Further, a reference B-mode frame may be fired between each ARFI frame and then a motion correction process may be applied for the ARFI frames using the reference that is closest in time. Alternatively, each ARFI frame may reference back to an older B-mode reference frame.

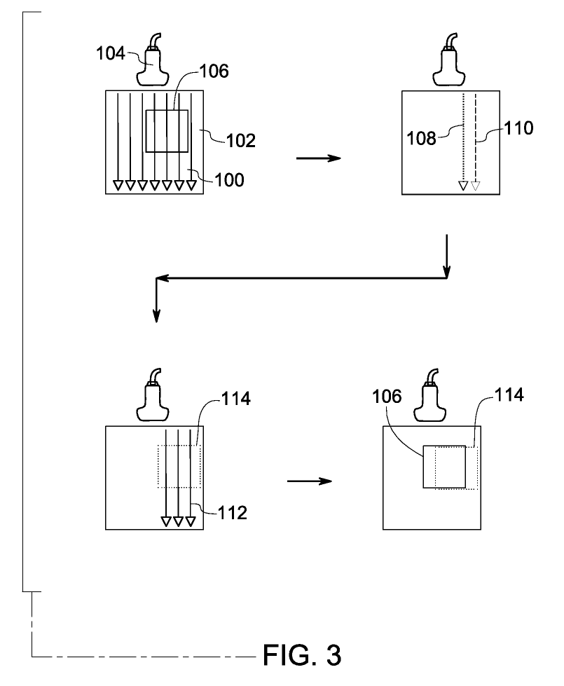

FIGS. 3, 4 and 5 illustrate an example of an application of a 2D cross correlation algorithm being applied to achieve motion correction. FIG. 3 illustrates a single frame 102 B-mode sequence 100 that is delivered to a region of interest 106 using a transducer probe 104. The B-mode image generated by the B-mode sequence 100 may be used as a reference image for alignment of the subsequent images. As will be appreciated, more than one pulse sequence may be delivered to a given location. The vector 108 represents a desired location for a representative pulse sequence in a series of firings being delivered to different locations in the frame 102, and the vector 110 represents an actual location for the pulse sequence 110. Next, a partial B-mode sequence 112 is delivered to the same region of interest 114 as the actual pulse sequence 110. The partial B-mode sequence 112 is used to estimate the location of the pulse sequence 110. As illustrated, there exists a shift in the actual and desired locations of the pulse sequences as represented by vectors 110 and 108, respectively. The region of interest 114 represents a region where the partial B-mode sequence 112 is delivered. To determine the displacement difference between the desired location of the pulse sequence 108 and the actual location of the pulse sequence 110 a cross correlation algorithm is used.

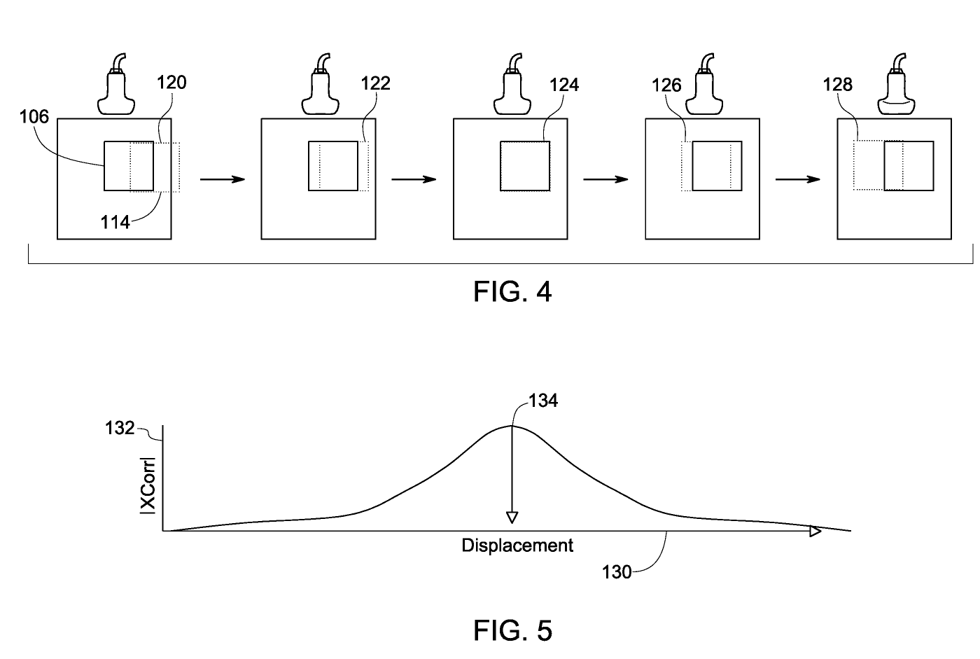

FIG. 4 illustrates an example application of a cross-correlation algorithm to determine the location of the region of interest 114 relative to the reference image region of interest 106. In the illustrated embodiments, the algorithm aligns the region of interest 114 over the initial B-mode image 100. At each location 120, 122, 124, 126 and 128 of the region of interest 114 in the initial B-mode image 100, the cross-correlation magnitude is calculated between the data from the partial B-mode 112 and the moving window of ROI 114 in the initial B-mode. The correlation magnitude is shown in FIG. 5. The abscissa 130 represents displacement of the region of interest 114, and the ordinate 132 represents the value of the correlation magnitude derived from applying the cross-correlation algorithm. The correlation magnitude peaks at the location 134 where the ROI 114 is best aligned with the B-mode data 106.

Although FIG. 4 shows the ROI 114 moving only from left to right, it should be appreciated that other movements of the ROI are also envisioned. For example, it is also possible to move the ROI up and down. In case of three-dimensional data it is possible to move the ROI in and out of plane.

Once the displacements have been determined for all of the vectors in the sequence, the scan conversion can interpolate an image in the plane using the actual locations of the vectors, thereby removing any distortion introduced by the motion.

In certain embodiments, a similar method as described in FIGS. 3-5 may be used to adjust the location of prospective firings based on the calculated location of previous firings. In this way, pulse sequence vectors may be more evenly distributed in the case of motion. Also, the motion of previous vectors may be used to predict the prospective motion and the adjustment of the firing locations may be made to compensate for the anticipated motion. An indicator of the quality of the pulse sequence data can be displayed on a display screen to provide a user with feedback about the image. This quality indicator may be based on correlation magnitudes of the pulse sequence tracking. The quality indicator may be based on the correlation magnitude of the motion compensation algorithm described in FIG. 5. Such a quality factor may be displayed to improve the users technique and to discard poor quality data.

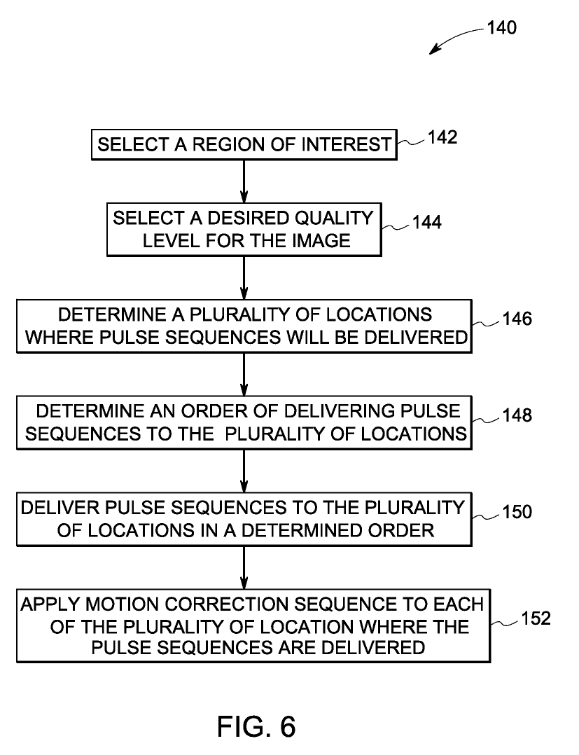

As mentioned above, in certain embodiments, the scan sequence may be modified to minimize the heating of the tissue in the region of interest. Delivering a pulse sequence repeatedly in the same direction results in increased tissue heating because all of the energy is deposited in the same location. However, delivering pulse sequences at spatially close locations near in time also can lead to increased tissue heating. Therefore, the scan sequence may be chosen to minimize the tissue heating. FIG. 6 is a flow chart 140 of an example of an algorithm that may be employed to reduce tissue heating in the region of interest. In the illustrated embodiment, the method begins by selecting a region of interest (block 142). The region of interest may be chosen by the operator, for example. At block 144, a desired quality level is selected for the image. While selecting the quality level a tradeoff may be made between the allowed thermal dose to the tissue and quality or type of information acquired from the image. The operator, such as the physician, may need to weigh the thermal dose and potential for damage against the possible benefit from the diagnosis. At block 146, a plurality of locations is determined within the region of interest for delivering the pulse sequences. For a given region of interest, the locations may be chosen based on the desired quality of the image.

At block 148, an order of delivering the pulse sequences to the plurality of locations is determined. The first push location of the determined order may be either chosen randomly, or may be a pushing location from a previous frame, or selected by the operator. The determined order may be based on a cost function that may be evaluated for each of the possible locations where the pulse sequence is to be delivered. The cost function may be designed to minimize the total thermal dose and the peak temperature rise. In one embodiment, the cost function is based on thermal models of the system. At block 150, the pulse sequences are delivered to each of the plurality of locations. The push location which minimizes the cost function (and therefore minimizes the thermal impact) is chosen as the next push location. Optionally, at block 152 based on thermal models or absolute rules, there may be cooling delay may be inserted at any point in the above scan sequence to ensure that the temperature rise is at an acceptable level. For example, if the cost function for the next position is higher than a threshold (i.e. if the thermal dose increases substantially with the next firing) then a cooling delay may be inserted by the algorithm. This process is repeated until the entire region of interest has been placed in the firing order. A cooling delay may be inserted by switching off the transducer probe between delivering two or more pulse sequences for example. At block 152, a motion correction sequence is applied to the plurality of locations. The motion correction sequence may be applied in a similar manner as discussed with regard to FIGS. 1-5.

In embodiments where multiple frames are to be imaged, the process illustrated in flow chart 140 is iterated through the subsequent frames. In some of these embodiments, the push locations may be moved to inter-grid locations to help reduce the heating at the peak. This shift can be accounted for in the scan conversion. This movement may reduce the total thermal dose.

In one embodiment, the cost function is based on a finite element model of the thermal spatiotemporal distribution. In this embodiment, the finite element model may model one or more of a transducer field, an ultrasound field, and a thermal distribution created by the ultrasound transmission. In another embodiment, relatively simpler ultrasound field models, which can be calculated more promptly, may be used as inputs to a finite element model which calculates the thermal distribution. The finite element model can model a simple homogenous material, or it can assume a typical configuration such a layer skin, a layer of fat, and a soft tissue layer, or it could be based on a complicated model made from an ultrasound, CT, MRI or other image.

In one embodiment, a simplified model may be used to determine the thermal cost of a firing. In this embodiment, it is assumed that the thermal dose delivered by a push pulse firing has a Gaussian spatial distribution in the lateral dimension. In the present embodiment, a model for the lateral distribution is provided for simplicity. Although the axial and elevational distributions may also be modeled. Assuming that the thermal distribution takes the form provided by Equation 1:

$$S ( x ) = {e}^{- \frac{{( x - {x}_{o} )}^{2}}{{σ}^{2}}} \qquad Equation 1$$

where, S(x) is the spatial variation in the thermal distribution, x is the lateral spatial coordinate, x₀ is the later position of the focus of the ultrasound pushing beam, and σ is the characteristic width of the thermal beam. σ is a function of the tissue and a function of the pushing pulse.

It is assumed that the temporal portion of the spatiotemporal distribution is modeled by an exponential decay of the form provided by Equation 2.

$$T ( t ) = {e}^{- \frac{t}{τ}} \qquad Equation 2$$

where, T(t) is the temporal variation of the thermal distribution, t is the time, and τ is a characteristic decay time which is a function of the tissue.

Further, assuming that the thermal contribution from a particular pushing pulse at a particular location and a given time is the product of the spatial and temporal factors.

D(x,t)=S(x)*T(t) Equation 3

Alternatively, assuming that the total thermal contribution at a particular spatial location and time is given by the sum of thermal contributions of all the previously fired pushing beams.

Given a set of vectors in a ROI, the firing order can be determined as follows. To begin, a first vector to fire is selected. D(x,t) values are calculated for each of the remaining possible vectors using Equation (3), where x is the location of the possible firing, and t is the current firing time. The sum of the D(x,t) values for each of the previously fired pushing vectors is determined, and the vector with the minimum sum is the next vector to be fired. In embodiments, where the sum of the D(x, t) values is greater than a threshold, then a delay may be introduced prior to firing the next pulse sequence.

Next, a determination is made as to which of the possible vectors has the minimum sum of the thermal contributions. The vector with the minimum sum will be the next vector to fire. If the sum is greater than a threshold, then a cooling delay is introduced prior to delivering the pulse sequence at the next location. The cooling delay may be determined such that the sum of D(x,t) for the new value of t is below the threshold value. This process is then repeated until all of the vectors in the ROI have been assigned to fire at a particular time.

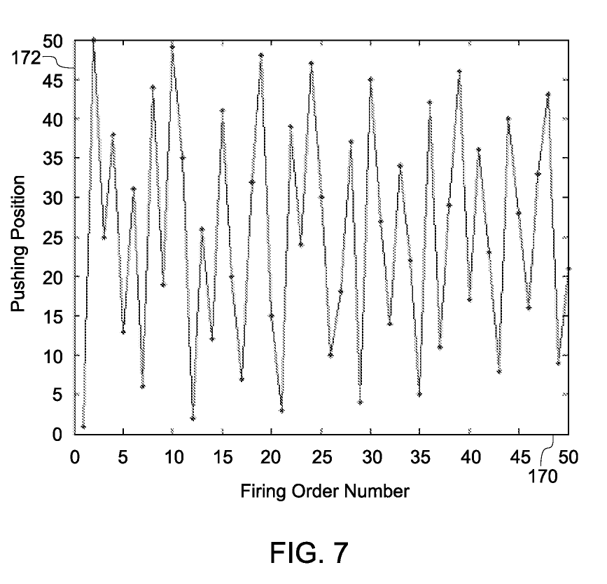

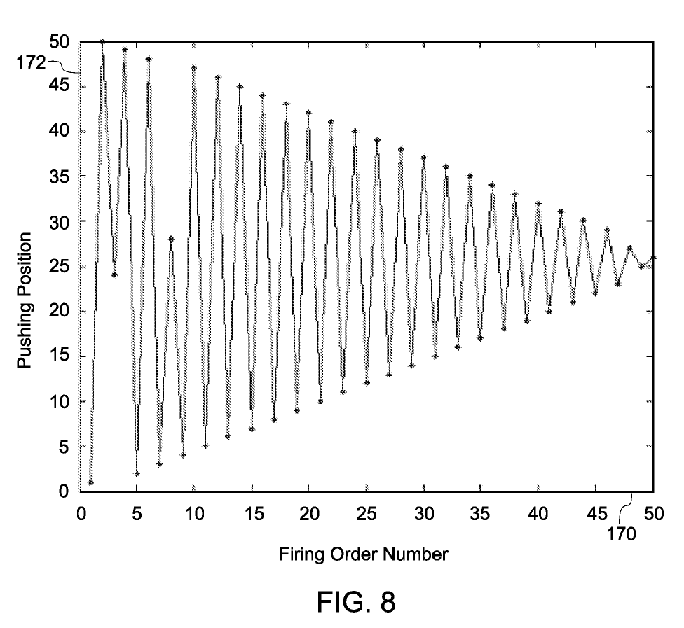

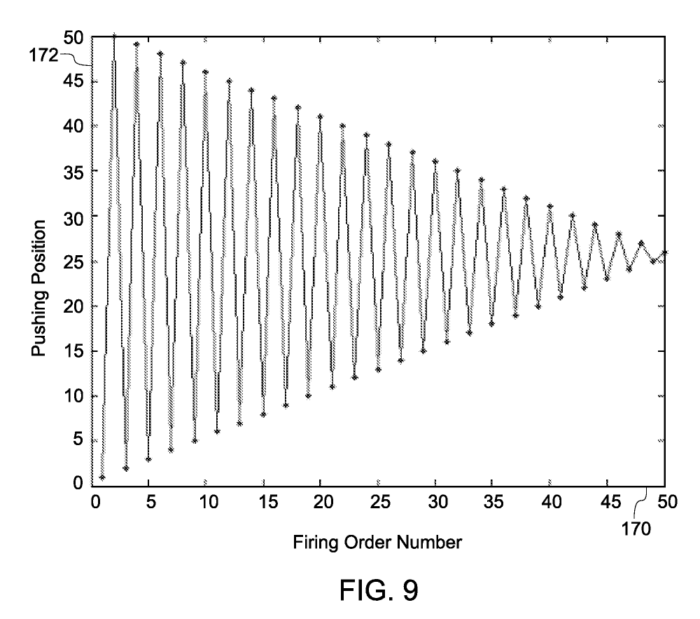

The spatial characteristic distance, σ, and the temporal characteristic time, τ, will typically effect the firing order. These values should be determined for the particular tissues and ultrasound beam parameters being used. FIGS. 7-9 illustrate examples of delivering the pulse sequences to the plurality of locations in a determined order. In the illustrated embodiments of FIGS. 7-9, the values of σ are varied. In the illustrated embodiment of FIG. 7, the value of σ is maintained at 5, while in FIG. 8, the value of σ is maintained at 25, and in FIG. 9, the value of σ is maintained at 50. The abscissa 170 represents the determined order of the pulse sequence, and the ordinate 172 represents the location to which the particular number of the pulse sequence is being delivered. In these embodiments, the plurality of locations to which the pulse sequences are to be delivered are separated by 1 distance unit and the time between delivering the pulse sequences to the plurality of locations is separated by 1 time unit. In these embodiments, τ is held constant at 10 time units. As illustrated in FIG. 7, for smaller values of σ (σ=5), that is for narrow width of the beam, the determined order is more varied. Whereas, as the value of σ is increased, that is, as the width of the beam is increased from 5 (FIG. 7) to 50 (FIG. 9), the determined order toggles between the extremes. The algorithm described may depend on the selection of the cost function to determine the order of delivering pulse sequences to the plurality of locations.

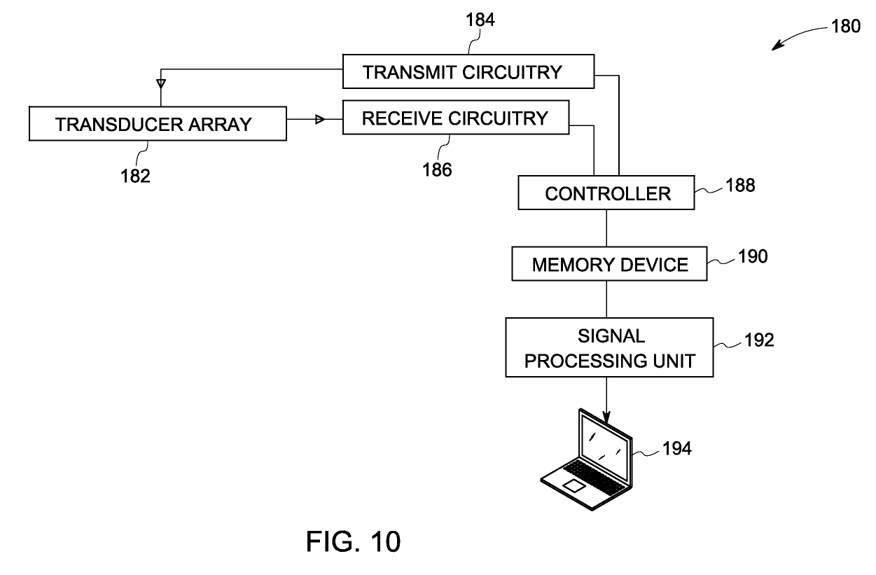

FIG. 10 illustrates an ultrasound imaging system 180 having a transducer array 182. The transducer array 182 may be a one-dimensional or a two-dimensional array. The transducer array 182 may be directed to a two-dimensional plane comprising one or more target regions. The reference pulse, pushing pulse, and the tracking pulse may be delivered using the transducer array 182. Typically, the transducer array 182 is in physical contact with the subject while delivering the pulses. Ultrasound imaging system 180 may further include transmit circuitry 184 and receive circuitry 186 in operative association with the transducer array 182 for respectively delivering the pulses, and receiving information from the plurality of locations where the pulse sequence is delivered. Both the transmit circuitry 184 and the receive circuitry 186 are electronically coupled to a controller 188. The controller 188 controls the pulse sequences including the time of delivery of the tracking pulse after the delivery of the pushing pulse and the delivery of the motion correction sequence. Further, the controller 188 may facilitate or enable indexing and storage of information received from the plurality of locations where the pulse sequence is delivered. The information received from the plurality of locations may be stored in the memory device 190 to be processed later in time. In one example, the memory device 190 may include a random access memory, however, other memory devices may be used. The memory device 190 may be used to store information such as initial position of the target region, and displaced position of the target region. A signal processing unit 192 then processes the information stored in the memory device 190. Alternatively, the signal-processing unit 192 may directly use the information from the controller 188 to generate images for the plurality of locations. The processed image is displayed using a display device 194, such as a monitor. Although not illustrated, a measuring device for point measurement of a displacement of the target region may be employed in place of the display device 194. Certain elements shown in FIG. 10 may be omitted or the functionality of certain elements may be combined with other elements. For example, the signal-processing unit 418 may be provided as part of the controller 188.

In some embodiments, one or more parameters of the pushing pulse or the tracking pulse may be altered from one location to another. In another embodiment, the parameters of the pushing pulse or the tracking pulse may be varied while delivering a subsequent pulse to the same location. In one embodiment, the one or more parameters that may be varied may include amplitude, a peak power, an average power, a length (length of the pushing pulse or the length of the pushing pulse packet), a frequency, a waveform, or combinations thereof. In another embodiment, the pulse repetition frequency (PRF) of the tracking pulse may be varied.

While only certain features of the invention have been illustrated and described herein, many modifications and changes will occur to those skilled in the art. It is, therefore, to be understood that the appended claims are intended to cover all such modifications and changes as fall within the true spirit of the invention.

1. An ultrasound imaging method, comprising:
identifying a plurality of locations within a region of interest;
delivering a pulse sequence to two or more of the plurality of locations in a determined order, wherein the pulse sequence comprises a pushing pulse, and a tracking pulse; and
applying a motion correction sequence to each of the plurality of locations where the pulse sequence is delivered.

2. The method of claim 1, wherein the method comprises imaging a plurality of frames.

3. The method of claim 2, wherein identifying the plurality of locations comprises selecting a first location of the plurality of locations based on a previous frame from the plurality of frames.

4. The method of claim 1, wherein delivering a pulse sequence to the two or more of the plurality of locations comprises determining the determined order based on a cost function for each of the plurality of locations, wherein the cost function is related to a total amount of heat provided to a location, or peak temperature for a location, or both.

5. The method of claim 4, comprising determining the cost function based on a finite element model.

6. The method of claim 1, further comprising introducing a cooling delay between two successive pulse sequences.

7. The method of claim 6, further comprising providing the cooling delay between the two successive pulse sequences if a value of a cost function for a next location is greater than a determined value.

8. The method of claim 1, further comprising imaging a plurality of frames such that the plurality of locations are inter-grid locations to facilitate reduction of thermal dose.

9. The method of claim 1, wherein applying the motion correction sequence comprises:
delivering an original frame B-mode sequence to a region of interest to obtain a reference image of the region of interest;
delivering a first pulse sequence to a first location in the region of interest;
delivering a first B-mode sequence overlapping the first location in the region of interest;
delivering a second pulse sequence to a second location in the region of interest; and
delivering a second B-mode sequence overlapping the second location in the region of interest; and
comparing images formed from the first and second pulse sequences with the reference image.

10. The method of claim 9, wherein delivering the first B-mode sequence, or the second B-mode sequence, or both comprises delivering the first B-mode sequence, or the second B-mode sequence immediately before or immediately after delivering the first pulse sequence, or the second pulse sequence, respectively.

11. The method of claim 9, wherein the first B-mode sequence or the second B-mode sequence comprises a partial B-mode sequence.

12. The method of claim 11, wherein a size of the partial B-mode is selected based on the determined level of the thermal dose, imaging time, or motion of tissues in at least one of the plurality of locations.

13. The method of claim 1, wherein the motion correction sequence employs at least one cross-correlation algorithm comprising 2D block matching, 3D block matching, 1D cross correlation, 2D cross correlation, 3D cross correlation, sum-of-absolute differences, sum of square difference, or minimum entropy.

14. The method of claim 9, further comprising applying a cross-correlation algorithm to determine a region of interest by aligning the region of interest over an original frame B-mode image.

15. The method of claim 1, further comprising predicting prospective involuntary motion of a transducer probe, a patient, a technician, or combinations thereof.

16. The method of claim 15, further comprising adjusting prospective deliveries of the pulse sequences based on the predicted involuntary motion.

17. An ultrasound imaging system; comprising:
a transducer array configured to deliver an ARFI pulse sequence to a plurality of locations in a region of interest, wherein the ARFI pulse sequence comprises a tracking pulse, and a pushing pulse;
a controller for controlling delivery of the ARFI pulse sequences to the plurality of locations in a determined order, or for controlling delivery of a motion correction sequence, and applying the motion correction sequence to each of the plurality of locations where the pulse sequence is delivered; and
a signal processing unit for processing received data from the plurality of locations in response to the plurality of ARFI pulse sequences, and the motion correction sequence.

18. The ultrasound imaging system of claim 17, wherein the plurality of locations is selected manually by operator, or selected employing an algorithm.

19. The ultrasound imaging system of claim 17, wherein the transducer array is a one-dimensional or a two-dimensional array.
